(19) United States
(12) Patent Application Publication
(10) Pub. No.: US 2011/0009739 A1
(43) Pub. Date: Jan. 13, 2011

(54) TRANSCRANIAL ULTRASOUND TRANSDUCER WITH STEREOTACTIC CONDUIT FOR PLACEMENT OF VENTRICULAR CATHETER

(76) Inventors: Scott B. Phillips, Detroit, MI (US); Satish Krishnamurthy, Manlius, NY (US)

Correspondence Address: John S. Economou, 202 Mamaroneck Ave., Third Floor, White Plains, NY, 10601, US

(21) Appl. No.: 12/833,144

(22) Filed: Jul. 9, 2010

Related U.S. Application Data
(60) Provisional application No. 61/224,985, filed on Jul. 13, 2009.

(51) Int. Cl. A61B 8/14; A61B 8/00

(52) U.S. Cl. 600/424; 600/463

(57) ABSTRACT

An apparatus, system and method for performing a ventriculostomy using an ultrasound probe is disclosed. A head portion of the probe is connected to a handle portion at a proximal end of the head portion. A transducer is mounted in a distal end of the head portion to transmit ultrasound waves into the patient. The head portion includes a conduit portion arranged to accept and to accurately direct a catheter into the body of the patient in a direction parallel to the direction of the ultrasound waves. The head portion is sized to fit into a conventionally-sized craniotomy. An adjustment mechanism connects the head portion to the handle portion for selectively adjusting the angle between the handle portion and the head portion. A sterile sheath having an integral conduit assembly for mounting to the head is optionally provided. An illuminator is optionally provided on the probe.

### CROSS-REFERENCES TO RELATED APPLICATIONS

The present application claims the benefit of the filing date of U.S. Provisional Patent Application No. 61/224,985, filed on Jul. 13, 2009, under 35 U.S.C. §119(e). U.S. Provisional Patent Application No. 61/224,985, filed on Jul. 13, 2009, is hereby incorporated by reference.

### FIELD OF INVENTION

The present invention relates to an apparatus, system and method for accurately positioning a catheter within a body cavity to be treated. More specifically, the disclosed inventive apparatus, system and method provide for the accurate introduction of a catheter into a targeted ventricular cavity of the brain during a ventriculostomy procedure.

### BACKGROUND OF THE INVENTION

A human brain includes four interconnected cavities, the ventricles, which produce and circulate cerebrospinal fluid. A ventriculostomy procedure involves the placement of a catheter into one of the ventricles. The ventriculostomy may be performed for intracranial pressure monitoring, draining or shunting of cerebrospinal fluid and/or the instillation of pharmacological therapeutic agents. A catheter functioning as an external ventricular drain is used to drain cerebrospinal fluid, and is typically done emergently to monitor and treat life-threatening hydrocephalus and/or intracranial hypertension. As a result, quick and accurate placement of the external ventricular drain is necessary.

The insertion of a catheter during a ventriculostomy was originally done completely by hand by the doctor based on external landmarks on the patient's body and on the doctor's knowledge of the internal three-dimensional anatomy of the brain which are used by the doctor to determine the trajectory of the insertion of the catheter into the targeted ventricle. External ventricular drain placement is a highly invasive procedure since the catheter passes through the brain parenchyma before entering the targeted ventricle and can cause iatrogenic hemorrhaging. In the freehand method, often multiple attempts (or passes) are required before the external ventricular drain enters the targeted ventricle, with each subsequent pass likely increasing the risk of iatrogenic hemorrhaging or other problems. For example, some external ventricular drains will inevitably fail to drain for various reasons. Most commonly, the external ventricular drain becomes obstructed by choroid plexus, clotted blood, proteinacious debris, or parenchymal debris. This may be remedied by flushing the catheter, but this can increase the risk of infection. If this and/or other troubleshooting measures fail, the external ventricular drain must be removed and replaced, necessitating an additional invasive procedure. As a result, greater accuracy during the initial placement of the external ventricular drain can decrease the frequency of obstruction by choroid plexus and parenchymal debris and therefore decrease the overall morbidity of an external ventricular drain. Thus, there is a clear need for an improved way to perform a ventriculostomy that provides such greater accuracy.

Another benefit of accurate placement of the external ventricular drain (i.e., the accurate placement of the catheter tip within the targeted ventricle) is that it broadens therapeutic options. Intraventricular tPA administration for intraventricular clotted blood may hasten patients' recovery from intraventricular hemorrhaging. In order to use intraventricular tPA, all of the eyelets in the catheter tip must be in the ventricle. This treatment is contraindicated when one or more of the eyelets are not in the ventricle but are instead in the parenchyma. Currently, there is no method of determining the location of the eyelets during the placement of an EVD. Although post-procedure head computed tomography (CT) can make such a determination and thus is the only conventional method of determining the viability of tPA therapy via the catheter placed within the ventricle, catheters cannot be repositioned after the procedure is completed and the skin is closed for risk of infection. Any repositioning” requires removal of the initial catheter and replacement by a second catheter and procedure.

One prior art approach to the need for accurate placement of the catheter tip within the ventricle uses a specially-designed catheter guide to help the doctor insert the catheter into the ventricle. The catheter guide is a tripod device placed on the skull and facilitates the creation of a trajectory for the catheter which is perpendicular to the tangent line of the skull. See, e.g., U.S. Pat. No. 4,613,324 to Ghajar, U.S. Pat. No. 4,821,716 Ghajar et al., U.S. Pat. No. 4,931,056 to Gharjar et al., U.S. Pat. No. 6,206,885 to Ghahremani et al. and U.S. Pat. No. 7,033,367 to Ghahremani et al. for several different catheter guides designed for this purpose. However, this approach is still “blind,” and is dependent on an accurately-positioned burr hole, a normocephalic skull shape and a non-distorted intracranial anatomy.

Another prior art approach provides an ultrasound probe within the tip of the catheter being inserted into the targeted ventricle. See, U.S. Pat. No. 5,690,117 to Gilbert, U.S. Patent Application Publication No. 2007/0083100 A1 to Schulz-Stubner and U.S. Patent Application Publication No. 2009/0088648 A1 to Jaffe et al. This approach requires a specially-designed and expensive catheter or stylet for performing the ventriculostomy. There are two other disadvantages. One is that the image quality from such a small transducer may be less than optimal. The second is that the perspective is from the tip of the catheter, thus there is no frame of reference provided to determine the depth of the placement. Essentially, this perspective provides “tunnel vision” and the operator would have to watch the target move towards the transducer tip (as opposed to a static image and target with the catheter seen moving into the ventricle).

Still another prior art approach is to use ultrasound to position a catheter within the targeted ventricle. In the article entitled “Accurate Placement Of Cerebrospinal Fluid Catheters With Real-Time Ultrasound Guidance In Older Children Without Patent Fontanels” by William E. Whitehead, M.D., et al. which appeared in Journal of Neurosurgery:Pediatrics, Volume 107 pp. 406-410 November, 2007 (the “Whitehead Article”) (incorporated herein by reference), the authors describe the use of a conventional ultrasound probe, Aloka Ultrasound Probe Model No. UST-5268P-5 (the “Aloka Probe”), attached to a conventional portable ultrasound machine (e.g., Aloka ProSound Model No. SSD-1700 II) with a conventional ventriculostomy catheter to guide the catheter. However, this probe is designed to fit within the 11 mm diameter spacing provided by a standard-size perforator drill bit, such as the Codman Disposable Perforator Model No. 26-1221, which generates a 14 mm upper diameter and an 11 mm lower diameter aperture through the skull of the patient. In addition, the Aloka Probe includes a shallow groove along one side thereof for use in guiding a needle biopsy probe into the brain. As a result, the method described in this article requires significant enlargement of the standard-sized burr hole to 15 to 20 mm to properly position both the catheter and probe against the dura in the burr hole opening, requiring either additional steps and the resultant extra time to perform the procedure or additional tools (i.e., additional drilling with multiple drill bits), and is not easily performed bedside. Another drawback is that although sterile probe covers are available for the Aloka Probe, these covers obscure the groove on the Aloka Probe and make it difficult to insert the catheter. As a result, it is likely that such covers will not likely be used, necessitating sterilization of the Aloka Probe after every use and a consequent delay in performing a subsequent procedure using the same Aloka Probe of between two to six hours for sterilization. A still further drawback of the use of the Aloka Probe is the need for the doctor to position the catheter within the shallow groove therein and hold it firmly against the Aloka Probe for the entire procedure. This additional effort generates fatigue and tremor in the doctor's hand and requires additional time for ensuring that the catheter is being properly inserted while performing the procedure.

Similarly, U.S. Patent Application Publication No. 2008/0287916 to Agmon discloses a clip 207 that is used to mate a conventional ultrasound probe 209 with a conventional catheter 205 to ensure that catheter 205 is guided to the targeted ventricle. Because the structures used to mate the catheter 205 to the probe 209 are not integral to the probe 209, less than optimal results are likely to occur. Furthermore, the use of a standard probe and standard catheter mated together would likely require a hole in the skull that is larger than that provided by the standard perforator drill bit, thereby either requiring a non-standard drill bit or multiple passes for creating the hole through the skull. Additionally, the guiding apparatus is fully enclosed and there is no mention of the guide being calibrated stereotactically towards the target. Finally, the use of a separate clip makes it difficult to employ a sterile sheath over the probe.

A ventriculostomy procedure is often done bedside in emergent situations, e.g., bedside in an ICU, and there is a need for an apparatus, system and method which ensures that this procedure is done accurately, e.g., without causing tissue damage, quickly, e.g., in a single pass and without requiring multiple pass drilling operations or complex drilling, and inexpensively, e.g., without requiring non-standard drill bits or other parts.

### SUMMARY OF THE INVENTION

The present invention is directed to an apparatus, system and method which overcomes the problems in the prior art. In particular, the present invention is an ultrasound probe comprising a handle portion for use in gripping the ultrasound probe and a head portion for directing ultrasound waves emitted from a transducer in a fixed direction into a body of a patient. The head portion is connected to the handle portion at a proximal end of the head portion such that an angle formed between the handle portion and the head portion is less than 150 degrees. The transducer is mounted in a distal end of the head portion. The head portion has a conduit portion on a periphery thereof arranged to accept a catheter therein and to direct the catheter into the body of the patient in a direction parallel to the direction of the ultrasound waves.

A cross-section of the head portion at the distal end in a direction perpendicular to the direction of the ultrasound waves may have a maximum width of less than 11 mm. The angle formed between the handle portion and the head portion may be approximately 90 degrees in one embodiment. The ultrasound probe may further comprising an adjustment mechanism for connecting the head portion to the handle portion for selectively adjusting the angle formed between the handle portion and the head portion from approximately 90 degrees to about 150 degrees. The conduit portion may be completely enclosed within the head portion or near-completely enclosed within the head portion. In another embodiment, the conduit portion may be attached on a periphery of the head portion adjacent to the proximal end thereof and the head portion may further comprise a channel in the periphery thereof adjacent to the distal end thereof, so that a catheter inserted through the conduit portion is guided to the channel portion and then into the body of the patient in a direction parallel to the direction of the ultrasound waves.

In a further embodiment, the conduit portion may include a first conduit portion fixedly attached to the head portion and a second conduit portion coupled to the first portion. The second conduit portion may be adapted to rotate into and out of an inner portion of the first conduit portion or may be removable.

A sterile sheath having an integral conduit assembly may be mounted over the head portion and the handle portion. A member attached as part of the conduit assembly is positioned on the inside of the sterile sheath and fits into a slot on the head portion to secure the conduit assembly against head portion. A conduit portion of the conduit assembly extends on the outside, sterile side, of the sterile sheath to provide a guide for insertion of the catheter.

In a still further embodiment, the conduit portion may detachable from the head portion via a snap fit female channel in the head portion and a correspond male portion in the conduit portion, or via matching snap fit connections at two edges of the head portion, or a circumferentially mounted snap fit connection. In addition, the conduit portion may be partially enclosed and snap fit stabilizing attachments may be used for securing the catheter within the conduit portion.

In a yet further embodiment, the ultrasound probe may further comprise an illumination device.

Furthermore, the present invention is directed to a system for positioning a catheter within a body of a patient, comprising an ultrasound machine and the ultrasound probe discussed above.

Finally, the present invention is directed to a method of inserting a catheter into a body of a patient using the ultrasound probe discussed above coupled to an ultrasound machine. The method comprises preparing an entry point for the catheter adjacent to a surface of a body of a patient, positioning the ultrasound probe against the entry point, inserting a catheter into the conduit portion of the ultrasound probe, guiding the catheter through the conduit portion into the body of the patient, and using a display coupled to the ultrasound machine to guide the continued insertion of the catheter to a targeted internal portion of the body. In particular, the method of the present invention includes a preparing step which comprises performing a craniotomy at a predetermined position on the patient and in which the entry point is a surface of the brain, the catheter is a ventriculostomy catheter and the targeted internal portion of the body is a ventricle within the brain.

### BRIEF DESCRIPTION OF THE DRAWINGS

The following detailed description, given by way of example and not intended to limit the present invention solely thereto, will best be understood in conjunction with the accompanying drawings in which:

FIG. 1 is a cross-sectional top view of the head of the ultrasound transducer probe of the present invention inserted within a conventional burr hole formed in a patient;

FIG. 2A is a side view of the ultrasound transducer probe of the present invention set to a first position, and FIG. 2B is a side view of the ultrasound transducer probe of the present invention set to a second position;

FIG. 3A is a perspective view of an embodiment of the head and integral conduit of the ultrasound transducer probe of the present invention, FIG. 3B is a perspective view of a further embodiment of the head and integral conduit of the ultrasound transducer probe of the present invention, and FIG. 3C is a perspective view of a still further embodiment of the head and integral conduit of the ultrasound transducer probe of the present invention;

FIGS. 4A, 4B and 4C show an embodiment of the head and partially rotatable conduit of the ultrasound transducer probe of the present invention;

FIGS. 5A, 5B and 5C show an embodiment of the head and partially removable conduit of the ultrasound transducer probe;

FIG. 6A is a perspective view of an embodiment of the head and snap fit conduit of the ultrasound transducer probe, FIG. 6B is a perspective view of a further embodiment of the head and snap fit conduit of the ultrasound transducer probe, FIG. 6C is a perspective view of a still further embodiment of a conduit assembly mounted within a slot in the head of the ultrasound transducer probe, and FIG. 6D is a perspective view of a yet still further embodiment of a conduit assembly of the ultrasound transducer probe showing snap fit attachments used to hold the catheter within the conduit;

FIG. 7A shows a sterile sheath assembly according to the invention having an integral conduit assembly for connection to the head portion of the ultrasound transducer probe, and FIG. 7B shows the sterile sheath assembly mounted on an ultrasound transducer probe; and

FIG. 8 is a side view of an ultrasound transducer probe of the present invention including an illumination device.

### DETAILED DESCRIPTION OF THE PREFERRED EMBODIMENT

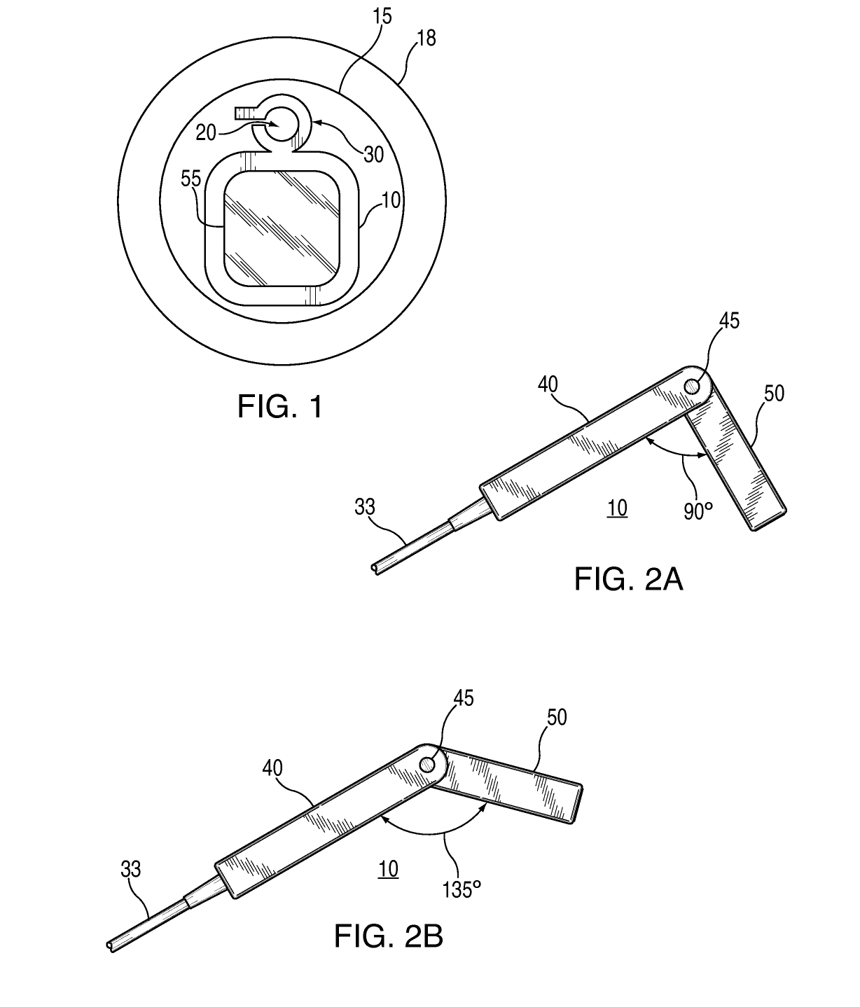

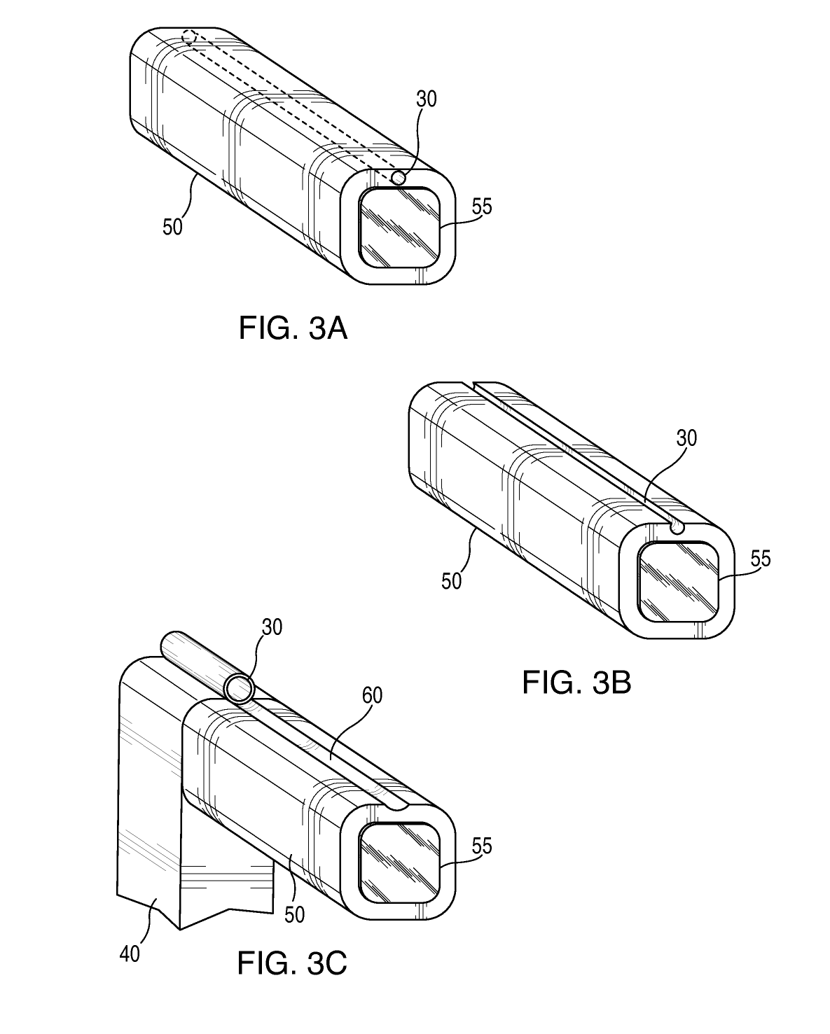

Referring now to the drawings and in particular FIG. 1, an ultrasound transducer probe 10 for use in guiding the placement of a ventriculostomy catheter via an aperture 20 in a stereotactic conduit 30 mounted on a side of probe 10. Transducer probe 10 is dimensioned to fit within an 11 mm diameter “burr hole” (craniotomy) 15 without requiring an additional step of increasing the diameter of the craniotomy. As discussed above, 11 mm is the current standard size inner cusp diameter craniotomy for ventriculostomies, and 14 mm is the current standard size outer cusp diameter 18. Transducer probe 10 is connected to a conventional portable ultrasound machine (not shown). Conduit 30 serves as a self-contained guide-port for probe 10 for insertion of a standard ventriculostomy catheter (not shown) via a cable 33 (FIG. 2). When the head of probe 10 is placed within the craniotomy and against the exposed surface of the brain (i.e., the dura), the alignment of the phased-array ultrasound waves emitted from a transducer 55 (see also FIGS. 3A to 3C) in the distal end of probe 10 and the guide-port are such that the area of the brain constituting the desired trajectory of the ventriculostomy catheter to the targeted ventricle may be seen on a monitor (not shown) of the portable ultrasound machine. In particular, the doctor inserting the ventriculostomy catheter may view the ventriculostomy catheter on the monitor as it is inserted, allowing a quicker and more accurate insertion of the catheter into the targeted ventricle.

As shown in FIG. 2A, transducer probe 10 preferably includes a handle 40 and a head 50 shown oriented at an angle of approximately 90 degrees to handle 40. Preferably, probe 10 includes an adjustment mechanism 45 so that this angle is adjustable to provide an angle from about 90 degrees to about 150 degrees, with the initial setting preset to an angle of 135 degrees (FIG. 2B). As one of ordinary skill in the art will readily recognize, the present invention is not limited to the use of an adjustment mechanism 45 and the angle between probe handle 40 and head 50 may be alternatively be preset to any angle between about 90 degrees and about 150 degrees. This configuration provides a much more ergonomic design than the design of the prior art Aloka Probe which has a bayoneted transducer in which the handle and probe head are oriented at an angle of approximately 180 degrees to each other (and the probe head bayoneted from the handle). This handle configuration allows the doctor to hold the probe 10 like a magnifying glass or joystick. The inventors have found that the configuration of the ultrasound probe 10 shown in FIG. 2 allows the doctor to stabilize his or her palm on the patient's head, thereby reducing fatigue and tremor while performing the ventriculostomy procedure and thereby providing better results.

As shown in FIGS. 3A, 3B and 3C, conduit 30 on probe head 50 is positioned and sized to ensure that a ventriculostomy catheter (not shown) is accurately inserted parallel and stereotactically aligned to the direction of the ultrasound waves so that any operator error with regards to trajectory variability of the catheter is minimized. In one embodiment shown in FIG. 3A, conduit 30 is completely enclosed within the probe head 50. In another embodiment shown in FIG. 3B, conduit 30 is near-completely enclosed within the probe head 50. In the embodiments shown in FIGS. 3A and 3B, the conduit 30 runs for the entire length of the probe head 50. In a still further embodiment shown in FIG. 3C, conduit 30 is completely enclosed and is attached perpendicularly to an end of probe head 50 adjacent to handle 40 and guides a ventriculostomy catheter (not shown) into a channel 60 on the probe head 50 to stabilize and maintain the catheter in proper alignment to stereotactic precision during insertion. Transducer 55 is coupled to the ultrasound machine via a cable 33 (FIGS. 2A and 2B).

By providing an enclosed or nearly-enclosed conduit 30 on probe 10, the effort required by the doctor in inserting the ventriculostomy catheter is significant reduced as opposed to performing the same procedure using the Aloka Probe discussed above and in the Whitehead Article. This result is obtained because the shallow groove on the Aloka Probe will contact less than about 25% of the circumference of the catheter and therefore requires the doctor to continuously and manually force and hold the catheter in place against the Aloka Probe during the insertion process. This makes the ventriculostomy procedure using the Aloka Probe prone to human error in alignment of the catheter trajectory to the targeted ventricle, creating a potential for failure of the procedure (necessitating an additional attempt or pass) and increasing the potential for harm to the patient. In the present invention, since conduit 30 is either completely or near-completely enclosed, the catheter is stabilized and maintained in alignment to stereotactic precision during insertion which greatly reduces human error and increases accuracy of the desired catheter trajectory because of the true stereotactic alignment of the conduit and the ultrasound waves.

The head 50 of ultrasound transducer probe 10 is shown in FIG. 2 with a nearly square cross-sectional head and in FIGS. 3A to 3C with a round cross-sectional head. The actual shape is arbitrary and may be selected for convenience of manufacture, with the only requirement being that head 50 and conduit 30 both fit into the inner-most area of a craniotomy created by a standard-size perforator drill bit, i.e., having a maximum outer diameter which allows the head to seat within the inner diameter (11 mm) of the craniotomy, is flush to the dura matter, and no part of the head 50 or conduit 30 are obstructed by the surrounding bone of the skull.

Although the conduit 30 may be either completely enclosed (FIG. 3A) or near-completely enclosed (FIG. 3B), in both cases a doctor must be able to remove probe 10 without disrupting the ventriculostomy catheter after insertion thereof into the targeted ventricle. As a result, when conduit 30 is completely enclosed as in FIG. 3A, probe 10 can only be removed by sliding it distally to the open (non-inserted) end of the ventriculostomy catheter. This may be impractical in certain situations because, e.g., cerebral spinal fluid could potentially leak onto the patient, doctor or the general area surrounding the patient; the newly-placed catheter could become dislodged; the movement of probe 10 could prove to be physically awkward; and the catheter could move into a non-sterile area or touch a non-sterile object while removing the probe 10. As one of ordinary skill in the art will readily recognize, when conduit 20 is partially enclosed as shown in FIG. 3B, the ventriculostomy catheter can be compressed along its central axis to slide it out of conduit 30 on probe 10. As a result, in this embodiment, there is no need to slide probe 10 distally along the ventriculostomy catheter.

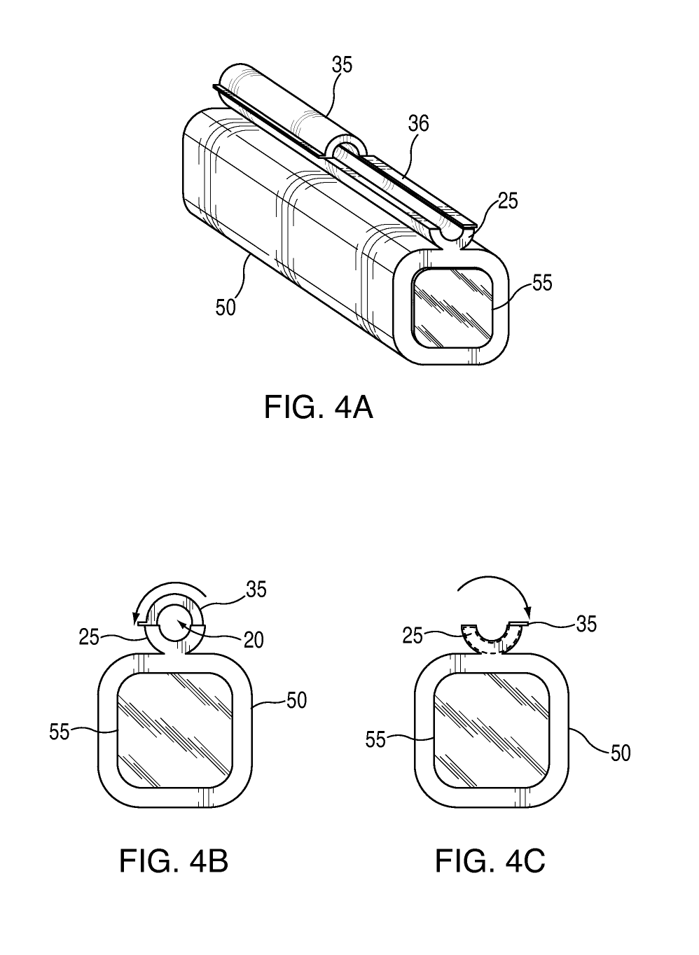

In one alternative embodiment shown in FIGS. 4A to 4C, conduit 30 on head 50 may be temporarily enclosed by configuring the outer half 35 of conduit 30 to roll (or rotate) into (and out of) the inner half 25 of conduit 30 to form, when closed, an aperture 20 for the catheter to move through. Thus the outer half is solid and the inner half is hollow and completely accepts the outer portion within it. As shown in FIG. 4A, conduit 30 may be constructed from two outer half portions 35 and 36 which each roll into (and out of) inner half 25. Alternatively, a single outer half 35 extending the length of inner half 25 may be provided or one of the outer half portions 35 and 36 (e.g., outer half portion 36) may be omitted. FIGS. 4B and 4C show the outer half 35 in the closed and open position, respectively.

After the ventriculostomy is completed and the catheter is inserted into the targeted ventricle, the outer half 35 of conduit 30 may be rotated into inner half 25 to enable easy removal of probe 10 without the drawbacks associated with removable by sliding probe 10 distally along the catheter.

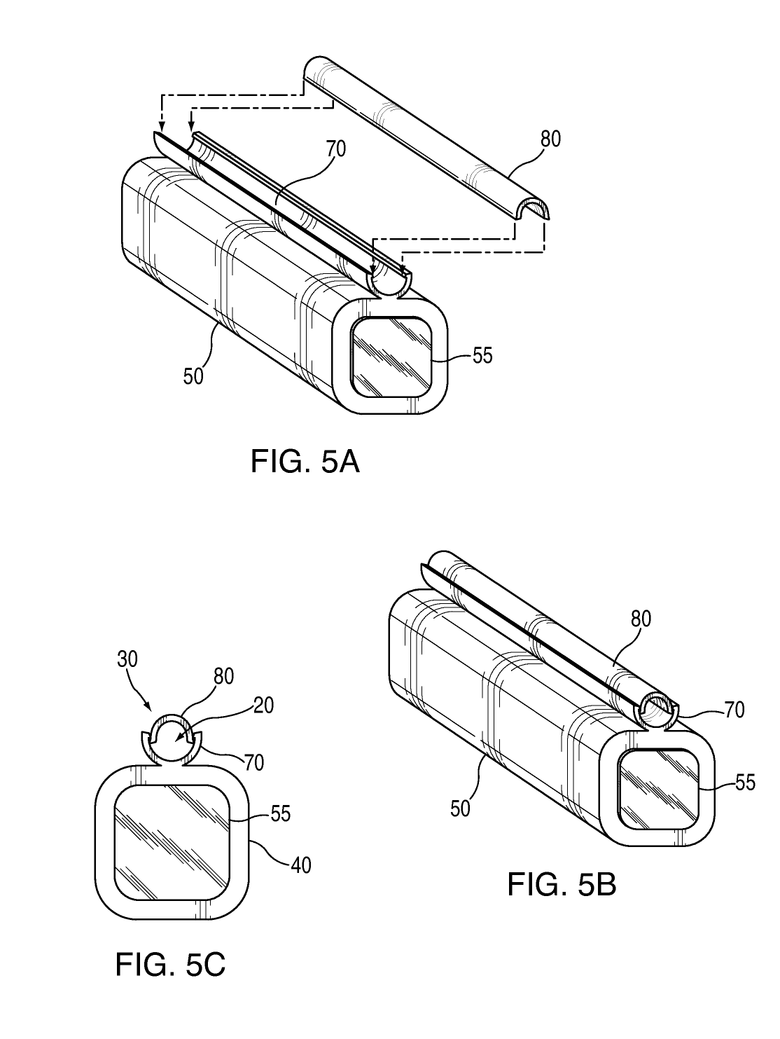

In another alternative embodiment shown in FIGS. 5A, 5B and 5C, conduit 30 on head 50 may be formed from a first part 70 forming a partial tube which is permanently affixed to head 50 and a second flexible part 80 which either slides or snaps onto part 70 to form fully-enclosed conduit 30 with an aperture 20 (FIG. 5C) for the catheter to move through. Flexible part 80 is made of a material (e.g., plastic) that is flexible enough to snap on and off (as shown in FIG. 5A), but rigid and attached firmly enough so that the set alignment of the system is maintained (FIG. 5B).

As with the prior embodiment, after the ventriculostomy is completed and the catheter is inserted into the targeted ventricle, the outer portion 80 of conduit 30 may be detached to enable easy removal of probe 10 without the drawbacks associated with removable by sliding probe 10 distally along the catheter.

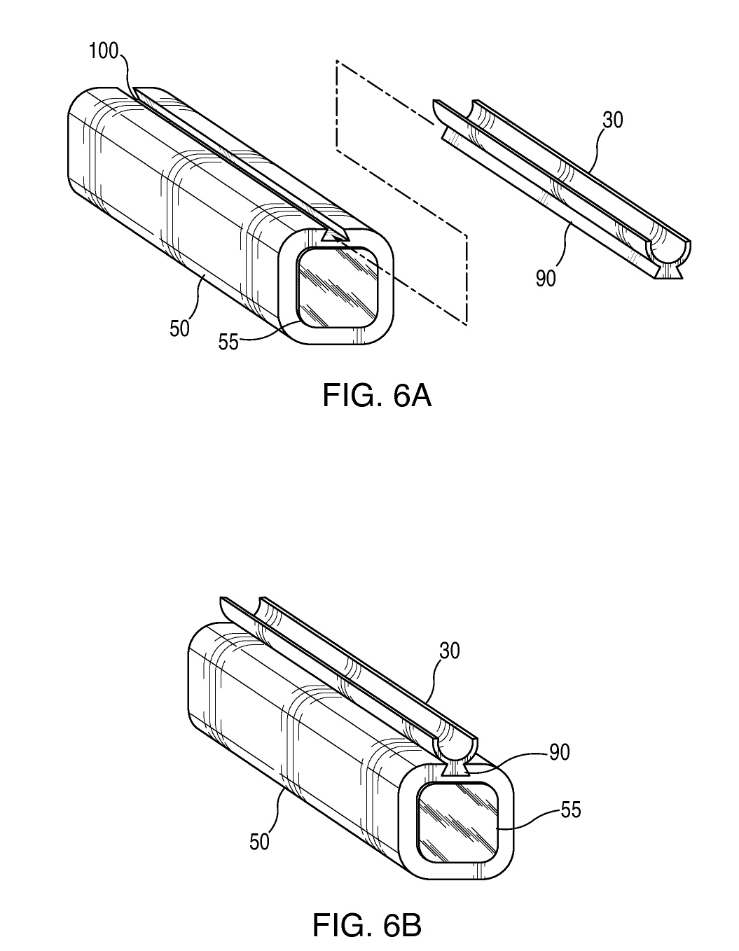

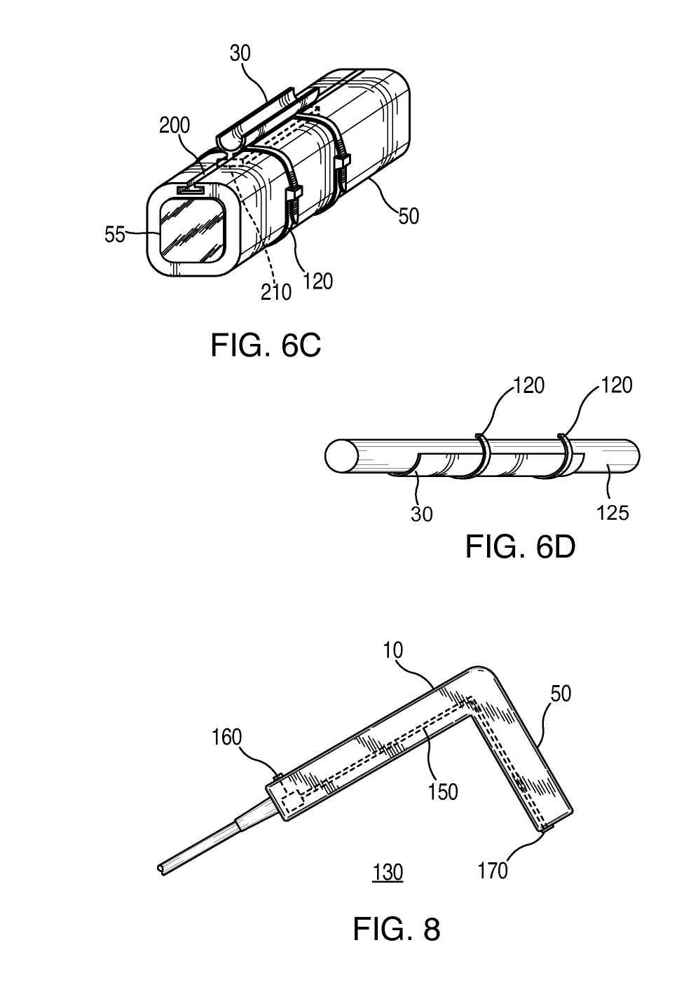

In a still further variation shown in FIGS. 6A to 6B, conduit 30 itself attaches in a “snap fit” (or sliding) manner to head 50. In FIG. 6A, conduit 30 is shown separated from head 50 and includes a male member 90 which fits into a female channel 100 on the head 50 to hold conduit 30 securely against head 50. In FIG. 6B, conduit 30 is shown mounted to head 50, with member 90 within channel 100.

In FIG. 6C, conduit 30 includes a flat member 210 which slides into slot 200. Conduit 30 is secured to head 50 in a conventional manner, e.g., with tie-wraps 120 which fit through a slot (not shown) on conduit 30. FIG. 6D shows snap fit attachments 120 for holding catheter 125 within a partially-enclosed conduit 30 (which itself may be attached to head 50 in any way discussed herein). Snap fit attachments 120 may be easily removed after placement of the catheter 125 to facilitate removal of the head 50 from the installed catheter 125.

Although there are commercially available sterile sheaths for use with the Aloka Probe so that it can be used repetitively on consecutive patients without the need for sterilization between each consecutive use, these sheaths take up more space in the operative field and obscure the groove in the side of the Aloka Probe. The ultrasound transducer probe 10 of the present invention allows a customized sterile sheath to be used with certain embodiments without any detrimental effect in the catheter insertion process, thereby negating any need for sterilization of the probe 10 between each procedure. For example, the embodiment shown in FIG. 6C designed such that a sterile sheath having an integral conduit 30 may be attached to head 50.

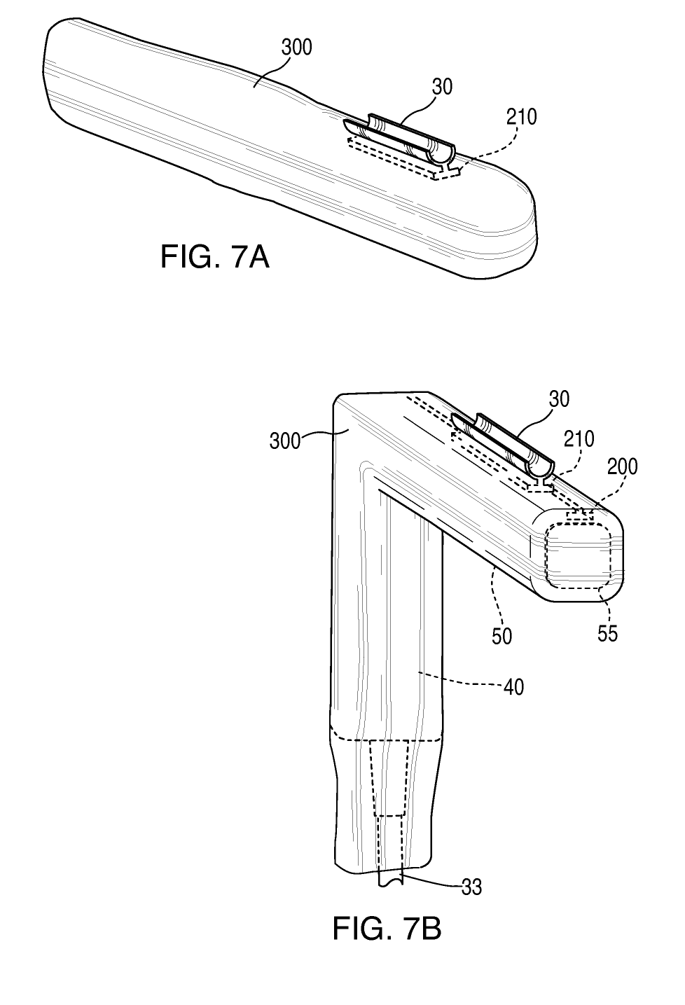

As shown in FIG. 7A, a sterile sheath 300 has a conduit assembly mounted therein having a conduit portion 30 on the outer, sterile side of sheath 300 and having a member 210 mounted on the inner, non-sterile side of sheath 300. Member 210 is adapted to fit into a corresponding slot 200 on head 50 as the sterile sheath 300 is placed over head 50, thereby securing the conduit assembly against head 50 and properly aligning conduit portion 30 for placement of the catheter. The sterile sheath 300 is shown mounted over the head 50, handle 40 and part of the cable 33 of ultrasound transducer probe in FIG. 7B. Member 210 is shown as an inverted-T structure which fits into a slot 200 on head 50, but any type of corresponding mating structures may be used, e.g., the snap fit connection shown in FIGS. 6A and 6B.

By employing a sterile sheath 300 having an integral conduit assembly, repeated sterilizations of the ultrasound transducer probe from procedure to procedure are not necessary, as only the removable sterile sheath 300 need to be replaced for each subsequent procedure. Furthermore, since all of the parts, i.e., the parts within the sheath that directly contact probe head 50 (and are within the non-sterile areas) and the parts outside the sheath that fall within the sterile areas are rigidly affixed to each other, the alignment of conduit 30 is maintained in stereotactic alignment with the phased-array ultrasound waves emitted from the head 50.

As discussed above in the Background of the Invention section, the Aloka Probe is too large to fit within the 11 mm diameter craniotomy hole created by the standard-sized perforator drill bit together with a ventriculostomy catheter and to resolve this problem, the authors of the Whitehead Article resorted to a 15 to 20 mm diameter craniotomy. By using the transducer head 10 shown in FIGS. 1 to 6, a doctor may perform a ventriculostomy using a standard-sized perforator drill bit (11 mm inner diameter/14 mm outer diameter) without the need for increasing the size of the craniotomy (as in the Whitehead Article).

As shown in FIG. 7, an illuminator 130 may preferably be added to probe 10 which allows additional light to be provided on the surface of the patient to aid the doctor in placement of the probe 10 and catheter 125. Illuminator 130 preferably includes a light source 140 coupled to a light tube 150 and is mounted integrally within probe 10 as shown in FIG. 7 or is attached to the periphery of probe 10 in a manner similar to conduit 30 as shown in FIGS. 6A to 6C. Illuminator 130 may include an on/off switch 160 mounted on the surface of probe 10 in a position easily accessed by the doctor performing the procedure. As one of ordinary skill in the art will readily recognize, light tube 150 may be mounted in a manner in which light is projected from the distal end of head 50 or in a manner in which light is projected from a point spaced away from the distal end of head 50 to assist in illuminating the area around the craniotomy when the probe 10 is placed against the surface of the patient. Illuminator 130 may also include a focusing lens 170 for directing the light in a predetermined pattern on the patient to additionally aid the doctor in the placement of the probe 10 and catheter 125. Also, the light tube 150 may be partially transparent and run along the surface of head 50 to further assist in lighting the area around probe 10.

The present invention provides a doctor with the ability to quickly and accurately insert a ventriculostomy catheter. The doctor first performs a craniotomy in the patient at the appropriate spot using the conventional burr-hole drill bit. Then the doctor positions probe 10 against the exposed dura in the 11 mm diameter hole created by the craniotomy. Probe 10 is coupled to an ultrasound machine to provide an ultrasound view of the brain. The doctor slides a conventional ventriculostomy catheter through the conduit 30 in probe 10. Conduit 30 accurately guides the ventriculostomy catheter in a direction parallel to the direction in which ultrasound waves are emitted from probe 10. As the ventriculostomy catheter enters the brain, the doctor is able to follow its progress on a display of the ultrasound machine. Probe 10 of the present invention is configured in an ergonomical manner so that the doctor can grip the handle 40 with one hand while guiding the ventriculostomy catheter with the other hand, thereby ensuring more accurate movement of the catheter towards the targeted ventricle.

While the present invention has been particularly shown and described with reference to the preferred embodiments and various aspects thereof, it will be appreciated by those of ordinary skill in the art that various changes and modifications may be made without departing from the spirit and scope of the invention. It is intended that the appended claims be interpreted as including the embodiments described herein, the alternatives mentioned above, and all equivalents thereto.

What is claimed is:

1. An ultrasound probe comprising:
a handle portion for use in gripping the ultrasound probe; and
a head portion for directing ultrasound waves emitted from a transducer in a fixed direction into a body of a patient, the head portion connected to the handle portion at a proximal end of the head portion such that an angle formed between the handle portion and the head portion is less than 150 degrees, the transducer mounted in a distal end of the head portion, the head portion having a conduit portion on a periphery thereof arranged to accept a catheter therein and to direct the catheter into the body of the patient in a direction parallel to the direction of the ultrasound waves.

2. The ultrasound probe of claim 1, wherein a cross-section of the head portion at the distal end in a direction perpendicular to the direction of the ultrasound waves has a maximum width of less than 11 mm.

3. The ultrasound probe of claim 1, wherein the angle formed between the handle portion and the head portion is approximately 90 degrees.

4. The ultrasound probe of claim 1, further comprising an adjustment mechanism for connecting the head portion to the handle portion for selectively adjusting the angle formed between the handle portion and the head portion from approximately 90 degrees to about 150 degrees.

5. The ultrasound probe of claim 1, wherein the conduit portion is completely enclosed within the head portion.

6. The ultrasound probe of claim 1, wherein the conduit portion is near-completely enclosed within the head portion.

7. The ultrasound probe of claim 1, wherein the conduit portion is attached on a periphery of the head portion adjacent to the proximal end thereof and further comprising a channel in the periphery of the head portion adjacent to the distal end thereof, whereby a catheter inserted through the conduit portion is guided to the channel portion and then into the body of the patient in a direction parallel to the direction of the ultrasound waves.

8. The ultrasound probe of claim 1, wherein the conduit portion includes a first conduit portion fixedly attached to the head portion and a second conduit portion coupled to the first portion.

9. The ultrasound probe of claim 8, wherein the second conduit portion is adapted to rotate into and out of an inner portion of the first conduit portion.

10. The ultrasound probe of claim 8, wherein the second conduit portion is formed from two parts each adapted to independently rotate into and out of an inner portion of the first conduit portion.

11. The ultrasound probe of claim 8, wherein the second conduit portion is removable.

12. The ultrasound probe of claim 1, wherein the conduit portion is detachable from the head portion via a snap fit female channel in the head portion and a correspond male portion in the conduit portion.

13. The ultrasound probe of claim 1, wherein the conduit portion is detachable from the head portion via matching snap fit connections at two edges of the head portion

14. The ultrasound probe of claim 1, wherein the conduit portion is detachable from the head portion via a circumferentially mounted snap fit connection.

15. The ultrasound probe of claim 1, wherein the conduit portion is partially enclosed and further comprising snap fit stabilizing attachments for securing the catheter within the conduit portion.

16. The ultrasound probe of claim 1, further comprising an illumination device.

17. A system for positioning a catheter within a body of a patient, comprising:
an ultrasound machine; and
an ultrasound probe coupled to the ultrasound machine comprising a handle portion for use in gripping the ultrasound probe and a head portion for directing ultrasound waves emitted from a transducer in a fixed direction into a body of a patient, the head portion connected to the handle portion at a proximal end of the head portion such that an angle formed between the handle portion and the head portion is less than 150 degrees, the transducer mounted in a distal end of the head portion, the head portion having a conduit portion on a periphery thereof arranged to accept a catheter therein and to direct the catheter into the body of the patient in a direction parallel to the direction of the ultrasound waves.

18. The system of claim 17, wherein a cross-section of the head portion at the distal end in a direction perpendicular to the direction of the ultrasound waves has a maximum width of less than 11 mm.

19. The system of claim 17, wherein the angle formed between the handle portion and the head portion is approximately 90 degrees.

20. The system of claim 17, further comprising an adjustment mechanism for connecting the head portion to the handle portion for selectively adjusting the angle formed between the handle portion and the head portion from approximately 90 degrees to about 150 degrees.

21. The system of claim 17, wherein the conduit portion is completely enclosed within the head portion.

22. The system of claim 17, wherein the conduit portion is near-completely enclosed within the head portion.

23. The system of claim 17, wherein the conduit portion is attached on a periphery of the head portion adjacent to the proximal end thereof and further comprising a channel in the periphery of the head portion adjacent to the distal end thereof, whereby a catheter inserted through the conduit portion is guided to the channel portion and then into the body of the patient in a direction parallel to the direction of the ultrasound waves.

24. The system of claim 17, wherein the conduit portion includes a first conduit portion fixedly attached to the head portion and a second conduit portion coupled to the first portion.

25. The system of claim 24, wherein the second conduit portion is adapted to rotate into and out of an inner portion of the first conduit portion.

26. The ultrasound probe of claim 24, wherein the second conduit portion is formed from two parts each adapted to independently rotate into and out of an inner portion of the first conduit portion.

27. The system of claim 24, wherein the second conduit portion is removable.

28. The system of claim 17, wherein the conduit portion is detachable from the head portion via a snap fit female channel in the head portion and a correspond male portion in the conduit portion.

29. The system of claim 17, wherein the conduit portion is detachable from the head portion via matching snap fit connections at two edges of the head portion.

30. The system of claim 17, wherein the conduit portion is detachable from the head portion via a circumferentially mounted snap fit connection.

31. The system of claim 17, wherein the conduit portion is partially enclosed and further comprising snap fit stabilizing attachments for securing the catheter within the conduit portion.

32. The system of claim 17, wherein the ultrasound probe further comprises an illumination device.

33. A method of inserting a catheter into a body of a patient using an ultrasound probe coupled to an ultrasound machine, the ultrasound probe comprising a handle portion for use in gripping the ultrasound probe and a head portion for directing ultrasound waves emitted from a transducer in a fixed direction into a body of a patient, the head portion connected to the handle portion at a proximal end of the head portion such that an angle formed between the handle portion and the head portion is less than 150 degrees, the transducer mounted in a distal end of the head portion, the head portion having a conduit portion on a periphery thereof arranged to accept a catheter therein and to direct the catheter into the body of the patient in a direction parallel to the direction of the ultrasound waves, the method comprising:
preparing an entry point for the catheter adjacent to a surface of a body of a patient;
positioning the ultrasound probe against the entry point;
inserting a catheter into the conduit portion of the ultrasound probe;
guiding the catheter through the conduit portion into the body of the patient; and
using a display coupled to the ultrasound machine to guide the continued insertion of the catheter to a targeted internal portion of the body.

34. The method of claim 33, wherein the preparing step comprises performing a craniotomy at a predetermined position on the patient, the entry point is a surface of the brain, the catheter is a ventriculostomy catheter and the targeted internal portion of the body is a ventricle within the brain.

35. A removable sterile sheath assembly for an ultrasound probe having a head portion and an engagement channel in the head portion, comprising:
a conduit assembly having a conduit portion and a member adapted to mount within the engagement channel in the head portion; and
a sterile sheath having an inner side and an outer side, wherein the conduit assembly is coupled to the sterile sheath such that the member is positioned adjacent to the inner side of the sterile sheath and the conduit portion is positioned adjacent to the outer side of the sterile sheath.

36. A system for guiding a catheter into a body, comprising:
an ultrasound probe having a handle portion and a head portion, the handle portion for use in gripping the ultrasound probe and the head portion for directing ultrasound waves emitted from a transducer in a fixed direction into a body of a patient, the head portion connected to the handle portion at a proximal end of the head portion such that an angle formed between the handle portion and the head portion is less than 150 degrees, the transducer mounted in a distal end of the head portion, the head portion having an engagement channel on a periphery thereof; and
a removable sterile sheath assembly having a conduit assembly, the conduit assembly having a conduit portion for guiding the catheter and a member adapted to mount within the engagement channel in the head portion and a sterile sheath having an inner side and an outer side, wherein the conduit assembly is coupled to the sterile sheath such that the member is positioned adjacent to the inner side of the sterile sheath and the conduit portion is positioned adjacent to the outer side of the sterile sheath.
